(19) United States
(12) Patent Application Publication
(10) Pub. No.: US 2026/0232335 A1
(43) Pub. Date: Aug. 13, 2026

(54) INTRAVASCULAR LITHOTRIPSY DEVICES AND SYSTEMS WITH CURRENT FAULT DETECTION

(71) Applicant: Cardiovascular Systems, Inc., St. Paul, MN (US)

(72) Inventors: Gregory B. Ingersoll, Minneapolis, MN (US); Matthew W. Tilstra, Rogers, MN (US)

(21) Appl. No.: 19/471,092

(22) PCT Filed: Mar. 29, 2024
(86) PCT No.: PCT/US2024/022239

§ 371 Date: Sep. 30, 2025

Related U.S. Application Data
(60) Provisional application No. 63/455,805, filed on Mar. 30, 2023.

(51) Int. Cl. A61B 17/22; A61B 17/00

(52) U.S. Cl. CPC A61B 17/22022; A61B 17/00234; A61B 2017/00039; A61B 2017/00238; A61B 2017/22025

(57) ABSTRACT

Intravascular lithotripsy (IVL) devices and fault detection of the electrical circuit including one or more emitters. A balanced current circuit includes a high voltage source, a control, and at least one emitter comprising a pair of electrodes across which a spark is to be generated. In a normal working condition, a closed loop electrical circuit with voltage applied via two conductors and current flowing between them when a spark is established. Under normal conditions, current IC on a first conductor from the control to the emitter is the same as current IR returning on a second conductor from the emitter back to the high voltage source and the circuit is in a balanced state. In a balanced state, there is no indication of current leakage from the circuit. In an unbalanced state, current IR is less that current IC, indicating an imbalance of the circuit that suggests a loss or leakage of a current IF.

### CROSS REFERENCE TO RELATED APPLICATIONS

This application claims priority to and the benefit of U.S. Provisional Patent Application No. 63/455,805, filed Mar. 30, 2023, the entire contents of which are incorporated herein by reference in their entireties.

### TECHNICAL FIELD

The present invention is directed to a catheter system for treating a vascular thrombus or calcified lesion or the like utilizing energy waves generated by electrodes within a conductive fluid medium.

### BACKGROUND

Catheter systems having an angioplasty balloon are commonly used to apply a physical force by expansion of the balloon against a calcified lesion within vasculature to force the calcification back into and against the blood vessel wall. Certain such calcified lesions and thrombi are not effectively broken up by the use of an angioplasty balloon.

More recently, catheter systems have been developed that include a balloon similar to an angioplasty balloon that is filled with a conductive liquid medium, such as a saline solution, for expanding the balloon in position at the lesion or thrombus, wherein the catheter system includes one or more pair(s) of electrodes operatively positioned within the conductive liquid medium. The electrodes are pulsed with high voltage direct current so as to create a spark that jumps over a gap between the two electrodes at each pulse. The spark within the conductive medium creates an energy wave that propagates through the liquid medium causing the balloon to physically provide a force against the lesion or thrombus. The energy propagation includes the creation of micro-bubbles that also facilitate the physical force. Such devices are known to provide an energy wave to act against a lesion or thrombus for purposes of breaking up the calcification or clotting.

Current catheter systems include a therapy sequence that includes a maximum number of continuous pulses, followed by a minimum delay time and a hard maximum total pulses associated with a particular catheter. One such product specifies:

| Treatment Frequency | 1 Hz (1 Pulse per Second) | |
|---|---|---|
| Maximum Number of Continuous Pulses | 30 | Pulses |
| (1 cycle) | | |
| Minimum Pause Time | 10 | Seconds |
| Maximum Total Pulses Per Catheter | 300 | Pulses |

If a therapy is not yet completed after the maximum total pulse per catheter count, the physician must replace the catheter with the attendant undesirable, cost, delay and distraction.

Intravascular lithotripsy (IVL) devices are available for some calcification patterns. Disposable IVL balloon devices are provided in different designs and sizes for peripheral or coronary indications. All designs utilize a reusable power source such as an IVL generator. One reusable DC generator comprises the following specification:

| | |
|---|---|
| Power | 110-240 VAC; 50-60 Hz; Single Phase, 15 A service |
| Size | 11″ (28.0 cm) high × 6″ (15.2 cm) wide × |
| | 11.5″ (29.2 cm) deep |
| Weight | 15 pounds (6.8 kg) |
| Output | Proprietary pulse delivery system. Output |
| | voltage 3000 volts peak, pulse frequency 1 Hz |
| Mobility | Product is designed to be mounted to an IV pole |
| Length | 5 ft (1.53 m) |
| Compatibility | Male key distally designed to connect only |
| | to catheter. |
| Operation | Lithotripsy pulsing is activated by pushing a |
| | button on the Connector Cable. |
| Use | Re-usable |

One such disposable device consists of a 0.014-inch guidewire-compatible, fluid-filled balloon angioplasty catheter with two lithotripsy emitters incorporated into the shaft of the 12-mm-long balloon segment. A fluid filled balloon (e.g. a 50/50 saline contrast medium) is inflated to about 4 atm and then electrical pulses are provided to the emitters that create high voltage sparks to provide the therapy. Acoustic waves are created and the calcium is fractured.

### SUMMARY

The present invention is directed to IVL devices and fault detection of the electrical circuit including one or more emitters (each emitter comprising a pair of spaced electrodes). A balanced current circuit includes a high voltage source, a control or console, and at least one emitter comprising a pair of electrodes across which a spark is to be generated. As above, electrical energy in the order of thousands of volts and tens or even hundreds of amps is used in Intravascular lithotripsy (IVL) devices. In a normal working condition, there is a closed loop electrical circuit with the voltage applied via two conductors and with current flowing between them when a spark is established. Under normal conditions, current IC on a first conductor from the console to the emitter is the same as current IR returning on a second conductor from the emitter back to the high voltage source. In this case, the circuit is in a balanced state. In such a balanced state, there would be no indication of current leakage from the circuit. In an unbalanced state of the circuit, a comparison of the current IC to the current IR shows a pulse over time difference in amplitude of the current over the pulse. Specifically, when current IR is less that current IC, there is an imbalance of the circuit that suggest a loss or leakage of a current IF.

In one aspect of the present invention, a method of determining a fault of an intravascular lithotripsy (IVL) device comprises a catheter with at least one emitter distal from a control module, a first conductor electrically running from the control module to and connected with the emitter, and a second conductor electrically running from and connected with the emitter to the control module, the first and second conductors electrically connected with a high voltage pulse generator by way of the control module as a system with the IVL device, wherein the method includes generating a high voltage pulse at the high voltage pulse generator; measuring a supply current though the first conductor and measuring a return current through the second conductor; and comparing the supply current to the return current to find any imbalance of the supply current to the return current.

Each emitter comprises an electrode pair spaced from one another for creating a spark across the electrode pair when a high voltage pulse is generated. In many cases, plural electrode pairs are provided electrically in series with one another. It is preferable then to perform the measuring step on the return current through the second conductor from a last electrode pair to the control module. In another preferred configuration, the measuring steps can be conducted within the control module.

In another preferred configuration, plural electrode pairs can be provided electrically in parallel with one another and with each of the parallel electrode pairs having a parallel portion of the first conductor, and further wherein the measuring of the supply current is done on the plural parallel portions of the first conductor.

The step of measuring the supply current can be done on the first conductor using a supply current sensor and the measuring of the return current is done on the second conductor using a return current sensor, and the comparing step can use the values obtained from each of the supply and return current sensors.

The measuring steps and comparing step can alternatively be conducted by passing the first conductor and the second conductor through a ferrous toroid so that current passing through the first conductor generates a current within the ferrous toroid in one direction while current passing through the second conductor generates a cancelling current within the ferrous toroid in an opposite direction with an imbalance indicated by current within the ferrous toroid in the one direction.

If it is determined that the supply current and the return current are not in an imbalanced state, but the measurement results indicate a reduced supply and return current as compared to measurement results over time, such a determination can indicate wear of an electrode pair.

The method can further use a difference amplifier circuit that receives measurement results of the supply current sensor and the return current sensor and amplifies the difference between them, the amplified difference then compared with a threshold value to determine any imbalance.

Alternatively, the method can further use a sense amplifier that receives a current value from the ferrous toroid and amplifies that current value, the amplified current value then compared with a threshold value to determine any imbalance.

In another aspect of the present invention, a system can determine a fault of an intravascular lithotripsy (IVL) device comprising a catheter with at least one emitter distal from a control module, a first conductor electrically running from the control module to and connected with the emitter, and a second conductor electrically running from and connected with the emitter to the control module, the first and second conductors electrically connected with a high voltage pulse generator by way of the control module as a system with the IVL device, the system being connectible with a high voltage pulse at the high voltage pulse generator for creating a spark at the emitter by way of the high voltage pulse, the system further comprising a sensor for measuring a supply current though the first conductor and a sensor for measuring a return current through the second conductor so that the supply current can be compared with the return current to find any imbalance of the supply current to the return current.

Each emitter comprises an electrode pair spaced from one another for creating a spark across the electrode pair when a high voltage pulse is generated. In many cases, plural electrode pairs are provided electrically in series with one another. It is preferable then to perform the measuring step on the return current through the second conductor from a last electrode pair to the control module. In another preferred configuration, the measuring steps can be conducted within the control module.

In such a system, plural electrode pairs can be provided electrically in parallel with one another and with each of the parallel electrode pairs having a parallel portion of the first conductor, and further wherein the sensor of the supply current is done on at least one of the plural parallel portions of the first conductor.

Specifically, the sensor for the supply current can be done on the first conductor and the sensing of the return current can be done on the second conductor, so that a comparison can use the values obtained from each of the supply and return current sensors.

In an alternative arrangement, the first conductor and the second conductor can be passed through a ferrous toroid so that current passing through the first conductor generates a current within the ferrous toroid in one direction while current passing through the second conductor generates a cancelling current within the ferrous toroid in an opposite direction with an imbalance indicated by current within the ferrous toroid in the one direction.

The system can further include a difference amplifier circuit that receives measurement results of the supply current sensor and the return current sensor and amplifies the difference between them, so that the amplified difference can then be compared with a threshold value to determine any imbalance.

Alternatively, the system can further use a sense amplifier that receives a current value from the ferrous toroid and amplifies that current value, so that the amplified current value can then be compared with a threshold value to determine any imbalance.

In yet another aspect of the present invention, a system of determining a fault of an intravascular lithotripsy (IVL) device can comprise a catheter with at least one emitter distal from a control module, the emitter comprises an electrode pair having electrodes spaced from one another for creating a spark across the electrode pair when a high voltage pulse is generated, the IVL device further comprising a first conductor electrically running from the control module to and connected with the emitter, and a second conductor electrically running from and connected with the emitter to the control module, the first and second conductors electrically connected with a high voltage pulse generator by way of the control module as a system with the IVL device, the system being connectible with a high voltage pulse at the high voltage pulse generator for creating a spark at the emitter by way of the high voltage pulse, the system further comprising a sensor for measuring a supply current though the first conductor and a sensor for measuring a return current through the second conductor so that the supply current can be compared with the return current to find any imbalance of the supply current to the return current, wherein either: (a) the sensor for the supply current is done on the first conductor and the sensing of the return current is done on the second conductor, so that a comparison can use the values obtained from each of the supply and return current sensors, or (b) the first conductor and the second conductor are passed through a ferrous toroid so that current passing through the first conductor generates a current within the ferrous toroid in one direction while current passing through the second conductor generates a cancelling current within the ferrous toroid in an opposite direction with an imbalance indicated by current within the ferrous toroid in the one direction.

### BRIEF DESCRIPTION OF THE DRAWINGS

FIG. 1 illustrates a system for providing intravascular lithotripsy according to an aspect of the present invention.

FIG. 2 illustrates an inflated balloon in a vessel for providing intravascular lithotripsy according to an aspect the present invention.

FIG. 3 schematically illustrates a current circuit for an IVL device in accordance with an aspect of the present invention with a first conductor and a second conductor in balance as to current flow.

FIG. 4 illustrates a graph of a high voltage pulse over time and showing a balanced circuit as in FIG. 3.

FIG. 5 schematically illustrates a current circuit for an IVL device in accordance with an aspect of the present invention with a first conductor and a second conductor unbalanced as to current flow.

FIG. 6 illustrates a graph of a high voltage pulse over time and showing an unbalanced circuit as in FIG. 5.

FIG. 7 illustrates a graph of a series of high voltage pulses over time and showing a balanced circuit over plural peaks followed by a detected imbalance.

FIG. 8 schematically illustrates a subcircuit of an aspect of the present invention for determining a balanced or unbalanced current within an IVL device circuit.

FIG. 9 illustrates a graph of a series of high voltage pulses over time and showing distinctions between a balanced circuit, wear to a balanced circuit, and a detected imbalance.

FIG. 10 is a schematic illustration of another current circuit for an IVL device in accordance with another aspect of the present invention with a first conductor and a second conductor passing through a ferrous toroid for detecting an imbalance to the circuit.

FIG. 11 is schematic current circuit for an IVL device similar to that of FIG. 10, but with plural conductors leading from the console to an IVL emitter.

FIG. 12 is schematic current circuit for an IVL device similar to that of FIGS. 10 and 11, but with plural circuits to and from an IVL emitter.

### DETAILED DESCRIPTION

The present invention is directed to IVL devices of the type that includes electrodes or lithotripsy emitters that create acoustic waves by arcing discharges between electrode components but may also include devices that create acoustic energy within the balloon via laser energy sources. Examples of such laser systems are described in U.S. Pat. Nos. 11,058,492 and 11,246,569 (the entire contents of which are incorporated by reference). Examples of electrically induced systems are described in U.S. Pat. Nos. 8,728,091, 9,642,673 and 10,850,078 and Published U.S. Pat. Appl. No. 2022-0054194 (the entire contents of which are incorporated by reference).

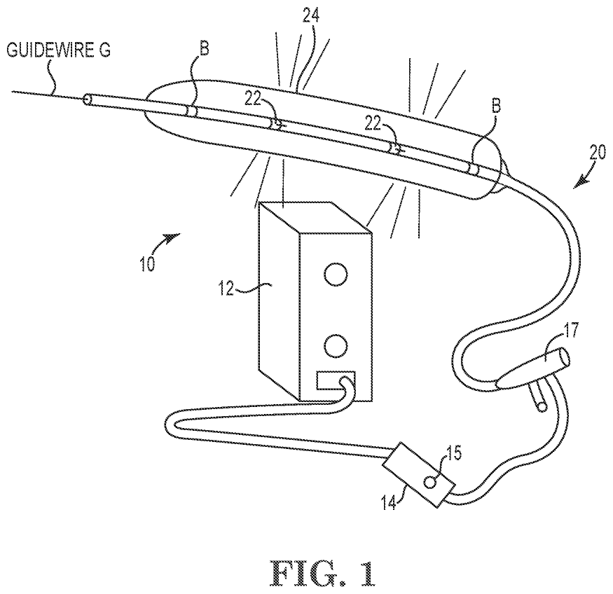

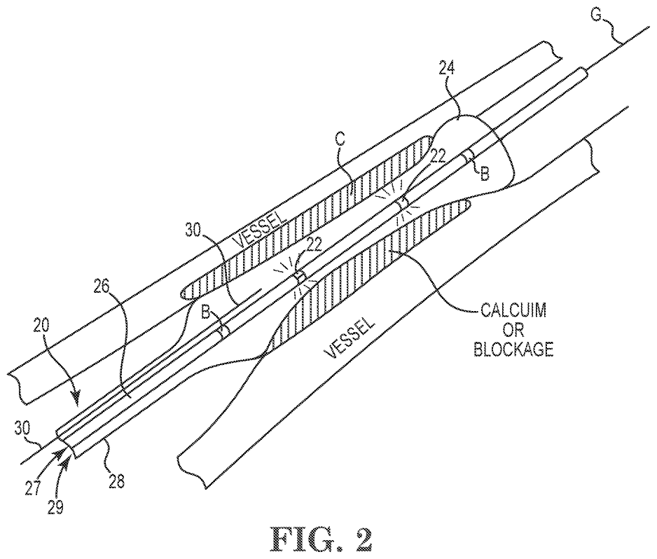

With reference to the Figures, FIGS. 1 and 2 show a system 10 according to the present invention comprising a console or power source 12 (in the form of an electrical generator, but alternatively in the form of a laser system), a handle 14 with therapy delivery control 15 and a catheter 20 with two lithotripsy emitters 22 (shown in the form of a pair of arcing electrodes, but alternatively they could comprise optical or laser emitters), and a fluid filled balloon 24. Optional marker bands B may be provided. The catheter 20 preferably includes a central tube 26 defining a guide wire lumen 27 through which a guide wire G passes for delivering the balloon 24 at the desired location along the guide wire G. A sheath 28 surrounds the central tube 26 and defines a delivery lumen 29 through which saline can be controllably delivered for balloon 24 inflation. The lumen 29 provides a concentric space around the central tube 26 within with electrode wires (not shown) can be run from the control 15 to the emitters 22 among other components in accordance with the present invention and discussed below. The sheath 28 is connected at a proximal end to a hub 17 that can include any number of ports allowing electrode wires to pass into the lumen 29 along with saline for inflation, the guide wire G, and any number of other components as desired.

The balloon 24 may be placed in a deflated position so as to more readily pass through a patient's vasculature to arrive at the scene of calcification. In use, the balloon 24 will be inflated to a common pressure for angioplasty procedures (e.g. 4 atm) and the therapy actuated via the delivery control 15.

FIG. 2 shows the balloon 24 inflated to a therapy delivery state where the lithotripsy emitters 22 may be “fired” to disrupt the vessel calcification C. Optional indicator bands B may be provided to afford visualization and proper positioning by use of known imaging techniques. The balloon 24 is inflated to a typical angioplasty pressure (e.g. 4 atm) and therapy is delivered. The balloon 24 may naturally expand during or just after the therapy is delivered to clear the vessel for passage of blood.

The control 15 is used to produce one or a series of voltage pulses in accordance with a treatment scheme. A high voltage pulse is provided to one of the emitters 22 comprising a pair of spaced electrodes and, in accordance with the illustrated embodiment, then in series to a second emitter 22 also comprising a pair of spaced electrodes. The high voltage pulse causes a spark across the first electrode pair then across the second electrode pair sequentially within the balloon 24. The somewhat conductive saline solution within the balloon 24 permits the high voltage spark across each electrode pair, thus creating an energy wave that propagates within the balloon toward the vessel calcification. As used for an IVL device of the present invention, a “high” voltage is preferably above 500V, more preferably above 1000V, and more preferably above 2000V.

Any spark created within a balloon 24 that is located within a patient's vasculature will also create a visible, or detectable, light event. Such light event can be detected at wavelengths other than that of visible light. Moreover, it is understood that a visible or detectable light leader can emanate from the ground connected electrode of any electrode pair when a high voltage pulse is initiated on the hot electrode pair prior to the actual spark event. Such a leader is similar to that seen to occur from conductive objects prior to a lightning strike. It is an object of the present invention to monitor these visible or detectable light events as feedback to the controlled generation of a high voltage spark. Understanding the timing of the creation of such a leader as well as the actual spark in one or more electrode pairs after generation of the high voltage pulse can lead to design variations of the electrode pairs as well as other components for the propagation of the energy waves within the balloon. It is a purpose of the present invention to better predict the timing of spark generation from when the high voltage pulse is sent by sensing any optical phenomenon that can predict when a spark will actually occur.

It is also contemplated that a catheter can be utilized in accordance with the present invention that does not include a balloon. Such a catheter would preferably include a lumen 29 that delivers saline to a controlled volume including the one or more emitters 22. Such a controlled volume can be created by structure of the vasculature of a patient along with the catheter distal end in the area of the emitters. Saline can be provided to fill such a controlled volume or may flow within and from such controlled volume at a controlled flow rate. A partial balloon is also contemplated from which saline fluid flow can weep from an open distal end of a partial balloon. Such a partial or open balloon design can be useful with a forward-facing electrode system such as disclosed within pending U.S. provisional patent application No. 63/416,231 filed Oct. 14, 2022, the entire contents of which are incorporated herein by reference. In the case of a catheter 20 having a balloon 24, the controlled volume is provided within the volume of the balloon 24.

As also shown in FIG. 2, an optical fiber 30 can be run within the lumen 29 to a desired point within the balloon 24 for the purpose of sensing the occurrence of light from a spark within the balloon 24. A proximal end of the optical fiber 30 is preferably optically connected with an optical detector that preferably provides an amplified signal of the sensed light. Optical fibers are well known as comprising an optical core within which light can propagate that is surrounded by a cladding layer. The cladding layer may further be surrounded by a protective layer. Light such as generated by a spark or precursor leader can travel along the optical core to be sensed by the optical detector, such as a photodiode. By directly detecting the spark or leader occurrence by the observed visible or detectable light (via the optical fiber), longevity of the IVL device can potentially be improved by varying the power usage for each spark and optimizing the spark by intensity thereof. It is contemplated that reducing the spark intensity and duration can benefit the longevity of the IVL and can reduce deleterious effects of heat that is generated by the spark. Also, control aspects and responsiveness can be improved by understanding spark intensity and timing relative to the high voltage pulses.

An additional optional use of the optical fiber 30 and an optical sensor is the detection of microbubbles within the saline solution of the balloon 24 that might interfere with passage of a shockwave. It is understood that micro-bubbles are created by the high voltage spark and resultant energy wave. It is also understood that such micro-bubbles can collapse under the application of certain energy to the micro-bubbles as suspended within a solution. Sonoluminescence is a phenomenon where the collapse of such a micro-bubble can result in both the generation of detectable light and sound waves. In the subject situation, a first pulse can create an energy wave and generate micro-bubbles with the saline solution within the balloon 24. A subsequent pulse can create another energy wave and micro-bubbles, but can also cause the collapse of micro-bubbles within the saline solution from the earlier pulse. Such an event can generate other detectable light that can be detected by the optical fiber 30. Such light generated from micro-bubble collapse might be detectable at a different wavelength that the spark detectable light, for example. Optical filters can be utilized depending on the optical phenomenon that is detected with an understanding of light emitted thereby at a specific wavelength. For example, sodium ion excitation in the saline solution can result is light emission in the 589 nm wavelength (yellow-orange) light. Detection of light at this wavelength could indicate properties of the spark and allow for adjustments to the spark generation.

In the case where one or more emitters 22 are fired by the control 15 and light is not detected by the optical fiber 30, this would be one example of a fault situation. Such a fault can result from a current leakage where current provided on the electrode wire heading to the one or more emitters 22 within the balloon 24 is different from (in this case more than) the current returning from the emitters 22 within the balloon 24 to the control 15. It is thus an aspect of the present invention to measure the current balance on the electrode wires to and from the emitters 22 to look for any current fault. In the case where current does not flow across an electrode gap between a pair of electrodes of an emitter and no spark is generated, the current flow from the emitter(s) 22 could be zero. This could mean that an emitter 22 is damaged or worn out, or that there is a current leakage elsewhere before the emitter 22. In the case where there is current flow across one or more emitters 22, regardless of sparking or not, a current difference or imbalance between the current suppled to the emitter and from the emitter can be a fault as well indicating current leakage somewhere along the circuit.

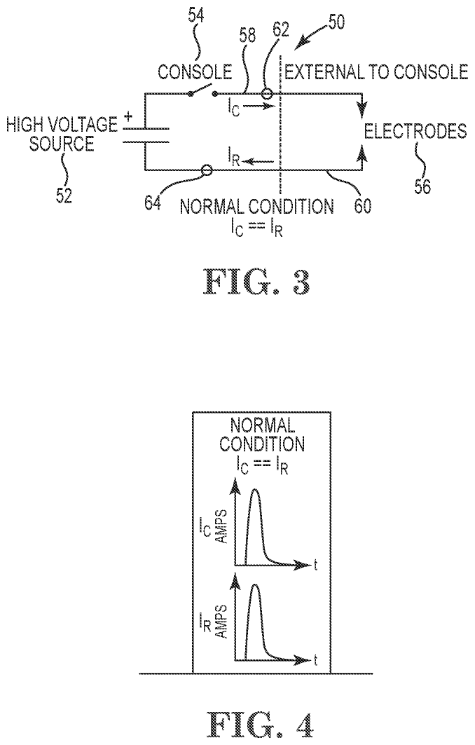

Referring now to FIGS. 3 and 4, a balanced current circuit 50 is shown including a high voltage source 52, a control or console 54, and an emitter 56 comprising a pair of electrodes across which a spark is to be generated. As above, electrical energy in the order of thousands of volts and tens or even hundreds of amps is used in Intravascular lithotripsy (IVL) devices. In a normal working condition, there is a closed loop electrical circuit 50 with the voltage applied via two conductors and with current flowing between them when a spark is established. Under normal conditions, current IC on a first conductor 58 from the console 54 to the emitter 56 is the same as current IR returning on a second conductor 60 from the emitter 56 back to the high voltage source 52. In the case, the circuit 50 is in a balanced state. In such a balanced state, there would be no indication of current leakage from the circuit 50. FIG. 4 shows a balanced state of the circuit 50 with the amps of the high voltage pulse over time and with the current IC equal to the current IR. It is understood that any circuit may have a slight variation from the IC current to the IR current, and a threshold value is preferably utilized within any comparison of the IC current to the IR current based on empirical information or otherwise.

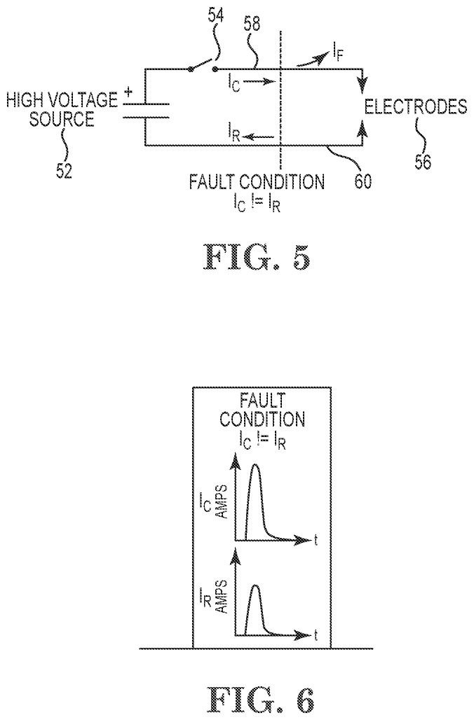

In FIGS. 5 and 6, the circuit 50 is illustrated with current IC from the console 54 to the emitter 56 and current IR from the emitter 56 back to the high voltage source 52. Under the occurrence of a spark being established at the emitter 56, IR and IC should be the same or substantially so, as above. However, as third current is illustrated as current IF that is a current leakage from the circuit 50 as can occur outside or inside of the console 54 into and through any other conductive medium. Such a current leakage IF can still allow a spark to occur at the emitter 56, but potentially with less energy and a reduced energy wave to work against a lesion, as above. FIG. 6 shows an unbalanced state of the circuit 50, wherein a comparison of the current IC to the current IR shows a pulse over time difference in amplitude of the current over the pulse. Specifically, current IR is less that current IC by the current IF in a generally sense taking into account small variations of the system.

Current passing through a conductor, such as conductor 58 and conductor 60 can be measured by any current sensor such as are commercially available. Examples include a current sensing resistor and a voltage amplifier for measuring the voltage differential. Other examples include a magnetic coil or a system that includes a Hall effect sensor. In one embodiment of a system according to the present invention and as schematically illustrated in FIG. 3, a first current sensor 62 is preferably provided along the first conductor 58 from the console 54 to the emitter 56 and a second current sensor 64 is preferably provided along the second conductor 60 from the emitter 56 to the high voltage source 52. In this way, current IC and current IR can be directly compared to one another. The current sensors 62 and 64 can be provided within the console 54 or anywhere along the length of the first and second conductors 58 and 60.

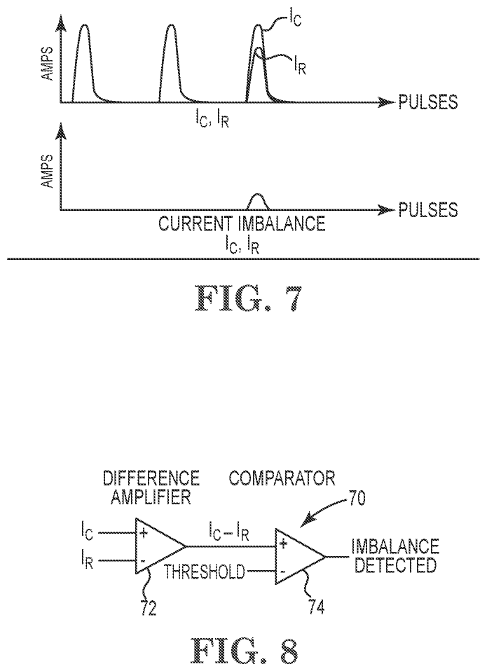

It is understood that with a system according to the present invention including current balance monitoring, that any such imbalance may be determined at any time during or after the firing of any number of high voltage sparks and energy waves. In FIG. 7, for example, three such high voltage pulses are illustrated over a time period with the first two pulses having balance current and a third pulse having a current imbalance. The first two pulses comprise the same curves as illustrated on top of one another, while the third pulse shows the curves distinctly from one another with the curve IR of less amplitude that the curve of IC with a greater amplitude. Such illustrates a current imbalanced, which amplitude of imbalance is illustrated in the lower graph below the third imbalanced pulse. In operation, such an imbalance within a series of pulses can thus control the ceasing of any further pulses once such an imbalance is determined. It is also contemplated that a pulse could even be terminated within the pulse if an imbalance is detected during the pulse.

FIG. 8 schematically illustrates an embodiment of a system subcircuit 70 that can be utilized for determining an imbalance based on a comparison of current IC with current IR. In this example, a current amplifier 72 receives current values from current sensors, such as 62 and 64, so as to amplify any difference between IC and IR. That difference is then provided to a comparator 74 that compares the amplified difference value to a threshold value. If the amplified difference value is greater than the threshold value, an imbalance is determined. Such amplifiers and comparators are well known and commercially available. It is understood that a comparator could be used alone without amplification and that a threshold value can be as low as zero and may be determined empirically or otherwise.

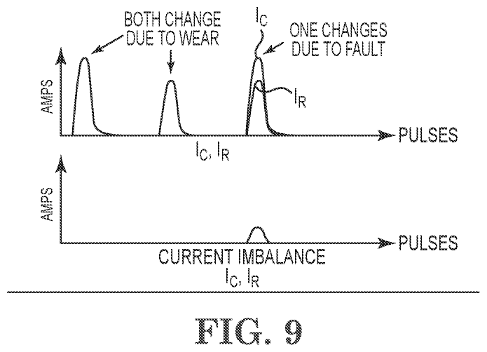

It is also noted that normal wear over time can be detected as well utilizing current sensors on both the first and second conductors 58, 60. As shown schematically in FIG. 9, the higher graph shows multiple pulses over time and the amplitude of both current curves representing both IC and IR during each pulse. The first pulse is as above with both curves on top of one another indication no imbalance or otherwise variations. The second pulse also shows both curves as the same on top of one another but of a lesser amplitude than the first pulse. In comparing the first and second pulses, presumably over some time period and any number of pulses in between them, the second pulse shows wear within the system (console or catheter) that increases resistance within the first and second conductors 58, 60. It is contemplated that comparing pulse amplitude over time of the multiple current sensors can provide feedback information of system wear or electrode wear that can also facilitate a determination of excessive wear of the system or electrodes requiring replacement. The third pulse illustrates an imbalance as described above with a amplitude difference as graphed below the third pulse.

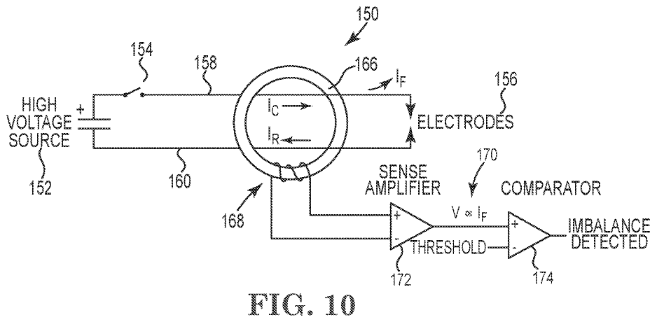

In another aspect of the present invention, the system can utilize a non-contact measurement to determine an imbalance. In the above systems, current sensors 62, 64 provide current data, amperage, at plural locations for comparison to one another. In FIG. 10, a circuit 150 is schematically illustrated including a high voltage source 152, conductors 158 and 160, an emitter 156 comprising an electrode pair, and a subcircuit 170 for making a determination of an imbalance. Instead of using current sensors, a ferrous toroid 166 can be provided so as to be operatively positioned for running the first and second conductors 158 and 160 through the open center of the ferrous toroid 166. As shown in FIG. 10, current IC running directionally from the console 154 to the emitter 156 passes in that direction through the open center of the toroid 166. Current IR running directionally from the emitter 156 to the high voltage source 152 passes in an opposite direction through the open center of the toroid 166. Current running in one direction through the center of the ferrous toroid 166 will generate a current in one direction within the ferrous toroid 166 via magnetic flux interaction. Current running in the opposite direction through the center of the ferrous toroid 166 will likewise generate a current in the opposite direction within the ferrous toroid 166. If both currents passing through the open center of the ferrous toroid are the same, no current within the toroid will occur. If there is a difference in the current within the first conductor 158 compared to the second conductor 160, a current will be generated within the ferrous toroid in one direction or the other based upon that difference. Any such difference can be the result of a leakage IF from the system and also indicated in FIG. 10. As above, the subcircuit 170 can first amplify at 172 any current generated within the ferrous toroid 166 and that value can be compared at 174 with a threshold value to determine an imbalance. As above, variations are contemplated.

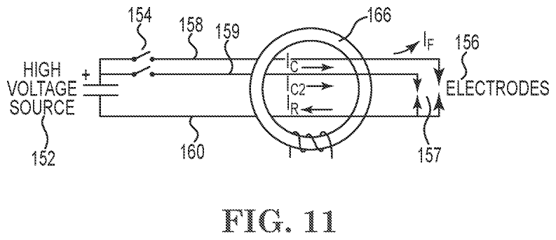

FIG. 11 shows a variation to the system of FIG. 10, wherein multiple conductors 158 and 159 run from the console 155 to a pair of emitters 156 and 157 in parallel to one another. Both emitters 156 and 157 are shown electrically connected to the high voltage source 152 by a return conductor 160. Multiple return conductors are also contemplated. In any case, even with the multiple conductors for current IC passing through the ferrous toroid 166, the current generation within the ferrous toroid 166 is the same as above.

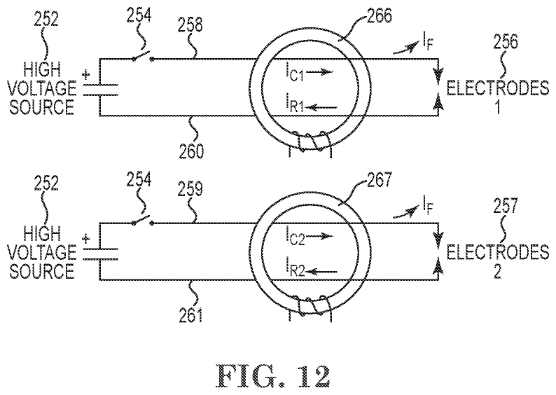

In contrast to the system of FIG. 11, a system 250 of FIG. 12 can include plural subsystems, each comprising a system circuit that is similar to that of FIG. 10. The subsystems can share a high voltage source 252 and console 254. Each subsystem can have their own first conductor 258, 259, running through a ferrous toroid 266, 267 to an emitter 256, 257 and a second conductor 260, 261 from an emitter 256, 257 to the high voltage source 252. Each ferrous toroid 266, 267 can then have an independent subcircuit (not shown) like subcircuit 170 for determining an imbalance. The advantage of the FIG. 12 system of the FIG. 11 system is the easier determination of the specific conductor and emitter from the other.

1. A method of determining a fault of an intravascular lithotripsy (IVL) device comprising a catheter with at least one emitter distal from a control module, a first conductor electrically running from the control module to and connected with the emitter, and a second conductor electrically running from and connected with the emitter to the control module, the first and second conductors electrically connected with a high voltage pulse generator by way of the control module as a system with the IVL device, the method comprising:
generating a high voltage pulse at the high voltage pulse generator;
measuring a supply current though the first conductor and measuring a return current through the second conductor; and
comparing the supply current to the return current to find any imbalance of the supply current to the return current.

2. The method of claim 1, wherein the emitter comprises an electrode pair spaced from one another for creating a spark across the electrode pair when a high voltage pulse is generated.

3. The method of claim 2, wherein plural electrode pairs are provided electrically in series with one another and the measuring step is done on the return current through the second conductor from a last electrode pair to the control module.

4. The method of claim 3, wherein the measuring steps are conducted within the control module.

5. The method of claim 2, wherein plural electrode pairs are provided electrically in parallel with one another and with each of the parallel electrode pairs having a parallel portion of the first conductor, and further wherein the measuring of the supply current is done on the plural parallel portions of the first conductor.

6. The method of claim 1, wherein the step of measuring the supply current is done on the first conductor using a supply current sensor and the measuring of the return current is done on the second conductor using a return current sensor, and the comparing step uses the values obtained from each of the supply and return current sensors.

7. The method of claim 1, wherein the measuring steps and comparing step are conducted by passing the first conductor and the second conductor through a ferrous toroid so that current passing through the first conductor generates a current within the ferrous toroid in one direction while current passing through the second conductor generates a cancelling current within the ferrous toroid in an opposite direction with an imbalance indicated by current within the ferrous toroid in the one direction.

8. The method of claim 2, wherein after the measuring steps it is determined that the supply current and the return current are not in an imbalanced state, but the measurement results indicate a reduced supply and return current as compared to measurement results over time, thus indicating wear of the electrode pair.

9. The method of claim 6, further comprising a difference amplifier circuit that receives measurement results of the supply current sensor and the return current sensor and amplifies the difference between them, the amplified difference then compared with a threshold value to determine any imbalance.

10. The method of claim 7, further comprising a sense amplifier that receives a current value from the ferrous toroid and amplifies that current value, the amplified current value then compared with a threshold value to determine any imbalance.

11. A system of determining a fault of an intravascular lithotripsy (IVL) device comprising a catheter with at least one emitter distal from a control module, a first conductor electrically running from the control module to and connected with the emitter, and a second conductor electrically running from and connected with the emitter to the control module, the first and second conductors electrically connected with a high voltage pulse generator by way of the control module as a system with the IVL device, the system being connectible with a high voltage pulse at the high voltage pulse generator for creating a spark at the emitter by way of the high voltage pulse, the system further comprising a sensor for measuring a supply current though the first conductor and a sensor for measuring a return current through the second conductor so that the supply current can be compared with the return current to find any imbalance of the supply current to the return current.

12. The system of claim 11, wherein the emitter comprises an electrode pair spaced from one another for creating the spark across the electrode pair when a high voltage pulse is generated.

13. The system of claim 12, wherein plural electrode pairs are provided electrically in series with one another and the sensor for measuring return current is done on the return current through the second conductor from a last electrode pair to the control module.

14. The system of claim 13, wherein the supply and return current sensors are conducted within the control module.

15. The system of claim 12, wherein plural electrode pairs are provided electrically in parallel with one another and with each of the parallel electrode pairs having a parallel portion of the first conductor, and further wherein the sensor of the supply current is done on at least one of the plural parallel portions of the first conductor.

16. The system of claim 11, wherein the sensor for the supply current is done on the first conductor and the sensing of the return current is done on the second conductor, so that a comparison can use the values obtained from each of the supply and return current sensors.

17. The system of claim 11, wherein the first conductor and the second conductor are passed through a ferrous toroid so that current passing through the first conductor generates a current within the ferrous toroid in one direction while current passing through the second conductor generates a cancelling current within the ferrous toroid in an opposite direction with an imbalance indicated by current within the ferrous toroid in the one direction.

18. The system of claim 16, further comprising a difference amplifier circuit that receives measurement results of the supply current sensor and the return current sensor and amplifies the difference between them, so that the amplified difference can then be compared with a threshold value to determine any imbalance.

19. The system of claim 17, further comprising a sense amplifier that receives a current value from the ferrous toroid and amplifies that current value, so that the amplified current value can then be compared with a threshold value to determine any imbalance.

20. A system of determining a fault of an intravascular lithotripsy (IVL) device comprising a catheter with at least one emitter distal from a control module, the emitter comprises an electrode pair having electrodes spaced from one another for creating a spark across the electrode pair when a high voltage pulse is generated, the IVL device further comprising a first conductor electrically running from the control module to and connected with the emitter, and a second conductor electrically running from and connected with the emitter to the control module, the first and second conductors electrically connected with a high voltage pulse generator by way of the control module as a system with the IVL device, the system being connectible with a high voltage pulse at the high voltage pulse generator for creating a spark at the emitter by way of the high voltage pulse, the system further comprising a sensor for measuring a supply current though the first conductor and a sensor for measuring a return current through the second conductor so that the supply current can be compared with the return current to find any imbalance of the supply current to the return current, wherein either: (a) the sensor for the supply current is done on the first conductor and the sensing of the return current is done on the second conductor, so that a comparison can use the values obtained from each of the supply and return current sensors, or (b) the first conductor and the second conductor are passed through a ferrous toroid so that current passing through the first conductor generates a current within the ferrous toroid in one direction while current passing through the second conductor generates a cancelling current within the ferrous toroid in an opposite direction with an imbalance indicated by current within the ferrous toroid in the one direction.
